(19) United States
(12) Patent Application Publication
(10) Pub. No.: US 2021/0259550 A1
(43) Pub. Date: Aug. 26, 2021

(54) Cadiovascular Monitoring Assembly

(71) Applicants: Bob Norri, Concord, CA (US); Rochelle Steele-Norri, Concord, CA (US)

(72) Inventors: Bob Norri, Concord, CA (US); Rochelle Steele-Norri, Concord, CA (US)

(21) Appl. No.: 16/796,502

(22) Filed: Feb. 20, 2020

(51) Int. Cl. A61B 5/00; A61B 5/024; A61B 5/021

(52) U.S. Cl. CPC A61B 5/0024; A61B 5/6832; A61B 5/024; A61B 5/6826; A61B 5/021; A61B 5/6822; A61B 5/6831; A61B 5/6823; A61B 5/6824; A61B 5/72

(57) ABSTRACT

A cardiovascular monitoring assembly for tracking cardiovascular health includes a set of transceivers that is configured for wireless communication and a set of detectors that is configured to couple to a user. The detectors are optical type. Each detector is configured to measure a respective cardiovascular parameter so that the set of detectors is configured to measure a variety of cardiovascular parameters. Each detector is operationally coupled to a respective transceiver so that the detector is positioned to relay a measurement of an associated cardiovascular parameter to the respective transceiver. The respective transceiver is configured to transmit the measurement to an electronic device. Processing programming code that is positioned on the electronic device enables the electronic device to display the measurements of the cardiovascular parameters on a display of the electronic device and to log the measurements in a data storage module of the electronic device.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to monitoring assemblies and more particularly pertains to a new monitoring assembly for tracking cardiovascular health.

### (2) Description of Related Art Including Information Disclosed Under 37 CFR 1.97 and 1.98.

The prior art relates to monitoring assemblies.

### BRIEF SUMMARY OF THE INVENTION

An embodiment of the disclosure meets the needs presented above by generally comprising a set of transceivers that is configured for wireless communication and a set of detectors that is configured to couple to a user. The detectors are optical type. Each detector is configured to measure a respective cardiovascular parameter so that the set of detectors is configured to measure a variety of cardiovascular parameters. Each detector is operationally coupled to a respective transceiver so that the detector is positioned to relay a measurement of an associated cardiovascular parameter to the respective transceiver. The respective transceiver is configured to transmit the measurement to an electronic device. Processing programming code that is positioned on the electronic device enables the electronic device to display the measurements of the cardiovascular parameters on a display of the electronic device and to log the measurements in a data storage module of the electronic device.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an in-use view of a cardiovascular monitoring assembly according to an embodiment of the disclosure.

FIG. 2 is an exploded view of an embodiment of the disclosure.

FIG. 3 is an isometric perspective view of an embodiment of the disclosure.

FIG. 4 is a top view of an embodiment of the disclosure.

FIG. 5 is a bottom view of an embodiment of the disclosure.

FIG. 6 is a block diagram of an embodiment of the disclosure.

### DETAILED DESCRIPTION OF THE INVENTION

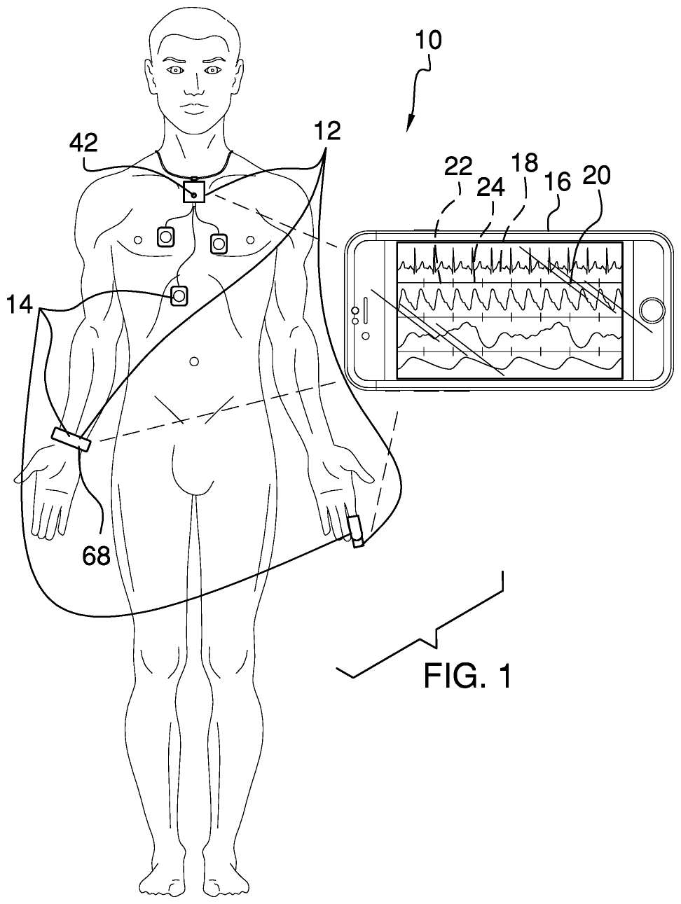

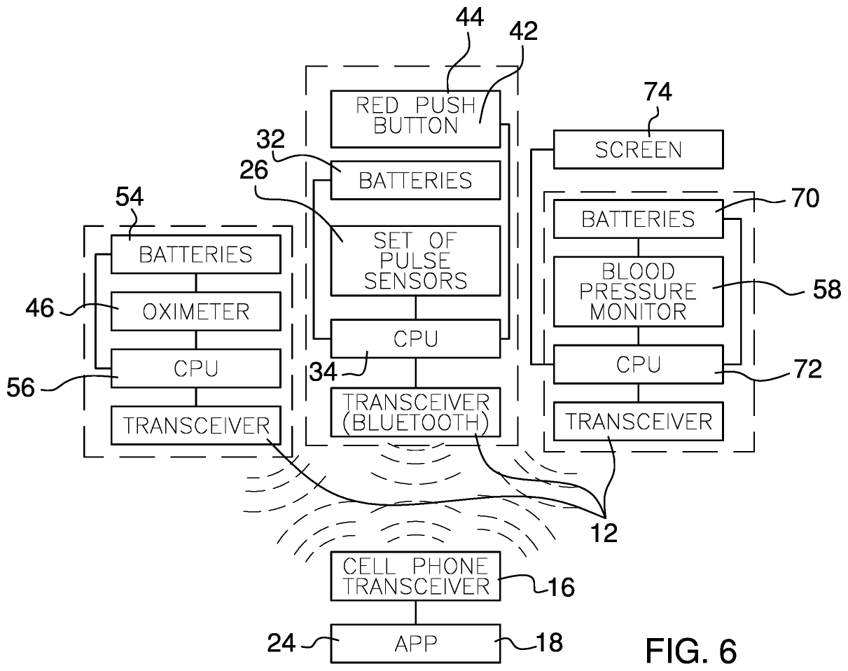

With reference now to the drawings, and in particular to FIGS. 1 through 6 thereof, a new monitoring assembly embodying the principles and concepts of an embodiment of the disclosure and generally designated by the reference numeral 10 will be described.

As best illustrated in FIGS. 1 through 6, the cardiovascular monitoring assembly 10 generally comprises a set of transceivers 12 that is configured for wireless communication and a set of detectors 14 that is configured to be coupled to a user. The detectors 14 are optical type. Each detector 14 is configured to measure a respective cardiovascular parameter so that the set of detectors 14 is configured to measure a variety of cardiovascular parameters. Each detector 14 is operationally coupled to a respective transceiver 12 so that the detector 14 is positioned to relay a measurement of an associated cardiovascular parameter to the respective transceiver 12. The respective transceiver 12 is configured to transmit the measurement to an electronic device 16, such as a smart phone, tablet computer, and the like.

Processing programming code 18 that is positioned on the electronic device 16 enables the electronic device 16 to display the measurements of the cardiovascular parameters on a display 20 of the electronic device 16 and to log the measurements in a data storage module 22 of the electronic device 16. The assembly 10 allows the user to view measurements of cardiovascular parameters in real time and to view graphical and animated representations of the cardiovascular parameters over time, which are derived from the logged measurements, on the display 20 of the electronic device 16.

Command programming code 24 that is positioned on the electronic device 16 enables the electronic device 16 to selectively signal the set of transceivers 12 to actuate the set of detectors 14 to obtain the measurements of the cardiovascular parameters. The command programming code 24 allows the user to capture the measurements of the cardiovascular parameters at any desired timepoint.

The set of detectors 14 comprises a set of first sensors 26 that is configured to be adhesively coupled to skin of the user. Each first sensor 26 is configured to measure a pulse rate of the user. The set of first sensors 26 may comprise three first sensors 26, or other number of first sensors 26, such as, but not limited to, one first sensor 26, two first sensors 26, and four first sensors 26.

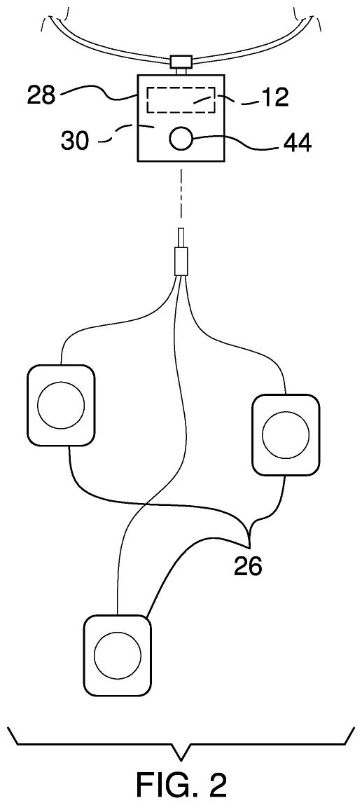

The set of first sensors 26 is wiredly coupled to a communications housing 28 that defines an interior space 30. The set of first sensors 26 may be removably couplable to the communications housing 28, as shown in FIG. 2. A communications battery 32, a communications microprocessor 34, and an associated transceiver 12 are coupled to the communications housing 28 and are positioned in the interior space 30. The communications microprocessor 34 is operationally coupled to the communications battery 32, the set of first sensors 26, and the associated transceiver 12 so that the communications microprocessor 34 is positioned to receive pulse rate measurements from the set of first sensors 26 and to actuate the associated transceiver 12 to transmit the measurements to the electronic device 16.

A strap 36 is coupled by opposing ends 38 to the communications housing 28 to define a loop 40. The strap 36 is configured to be positioned around a neck of the user, as shown in FIG. 1, so that the communications housing 28 is positioned proximate to a sternum of the user and so that the set of first sensors 26 is positioned to be adhesively coupled to the chest of the user. The present invention anticipates other means to define the loop 40, such as, but not limited to, cords, chains, and the like.

A switch 42 that is coupled to the communications housing 28 is operationally coupled to the communications microprocessor 34. The switch 42 is configured to be switched to signal the communications microprocessor 34 to denote a pulse rate measurement of interest, for example a measurement taken when the user is experiencing chest pain. The switch 42 may comprise a button 44, as shown in FIG. 2, which is depressible, or other switching means, such as, but not limited to, toggles, slide switches, and the like. The button 44 may red colored.

The set of detectors 14 also comprises a second sensor 46 that is configured to be coupled to an extremity of the user, such as a digit of a hand of the user and an ear of the user. The second sensor 46 is configured to measure a blood oxygenation level of the user.

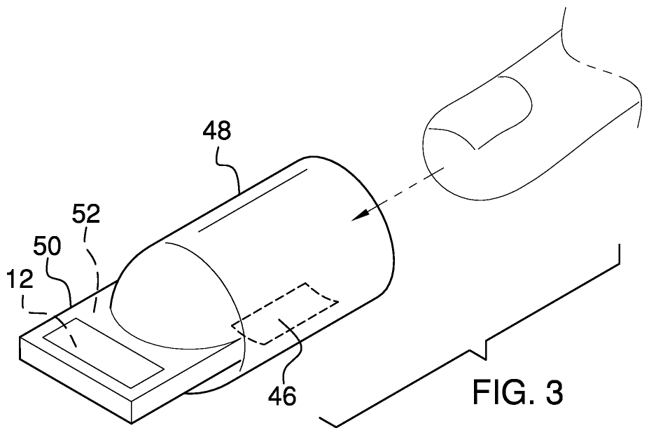

The second sensor 46 may be coupled to a sleeve 48 that is configured to insert the digit of the hand of the user, as shown in FIG. 3. The second sensor 46 is configured to contact skin of the digit of the hand as the digit is inserted into the sleeve 48. The second sensor 46 also may be coupled to a clip (not shown) that is configured to be coupled to an ear of the user.

A transmissions housing 50 that is coupled to the sleeve 48 defines an internal space 52. A transmissions battery 54, a transmissions microprocessor 56, and an associated transceiver 12 are coupled to the transmissions housing 50 and are positioned in the internal space 52. The transmissions microprocessor 56 is operationally coupled to the transmissions battery 54, the second sensor 46, and the associated transceiver 12 so that the transmissions microprocessor 56 is positioned to receive the measurement of the blood oxygenation level from the second sensor 46 and to actuate the associated transceiver 12 to transmit the measurement to the electronic device 16.

The set of detectors 14 also comprises a third sensor 58 that is configured to be coupled to at least one of a limb and the digit of the hand of the user. The third sensor 58 is configured to measure a blood pressure of the user.

The third sensor 58 is coupled to a lower face 60 of a cuff housing 62, as shown in FIG. 5. The cuff housing 62 defines an inner space 64. The cuff housing 62 and a band 66 together comprise a wrist cuff 68. The band 66 is configured to couple the cuff housing 62 to a wrist of the user so that the third sensor 58 is configured to contact skin of the wrist of the user. A cuff battery 70, a cuff microprocessor 72, and an associated transceiver 12 are coupled to the cuff housing 62 and are positioned in the inner space 64.

The cuff microprocessor 72 is operationally coupled to the cuff battery 70, the third sensor 58, and the associated transceiver 12 so that the cuff microprocessor 72 is positioned to receive the measurement of the blood pressure from the third sensor 58 and to actuate the associated transceiver 12 to transmit the measurement to the electronic device 16.

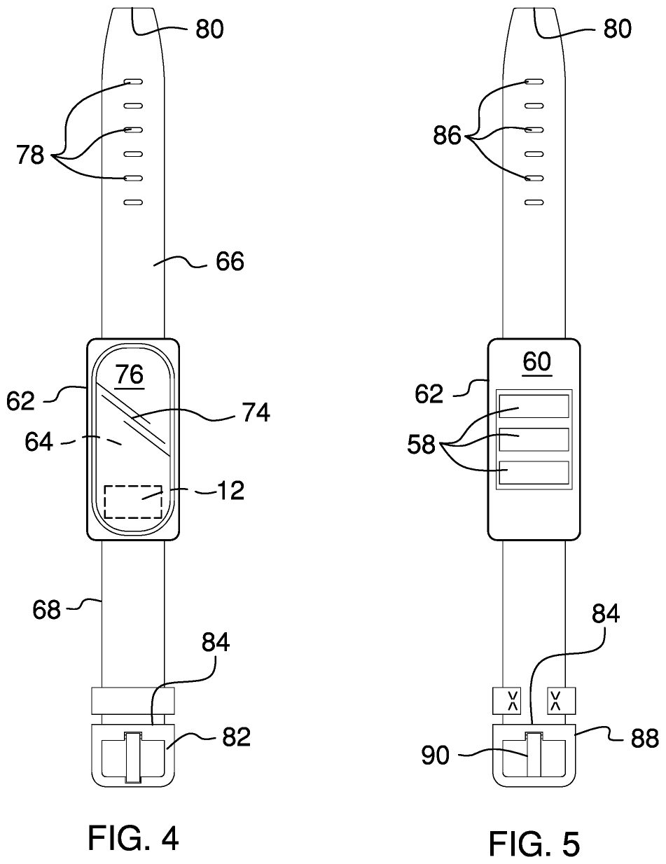

A screen 74 is coupled to an upper face 76 of the cuff housing 62, as shown in FIG. 4. The screen 74 is operationally coupled to the cuff microprocessor 72 so that the cuff microprocessor 72 is positioned to selectively actuate the screen 74 to display a time and the measurement of the blood pressure.

A first fastener 78 is coupled to the band 66 proximate to a first end 80 of the band 66. A second fastener 82 is coupled to the band 66 proximate to a second end 84 of the band 66. The second fastener 82 is complementary to the first fastener 78 so that the second fastener 82 is positioned to selectively couple to the first fastener 78 to couple the band 66 around the wrist of the user. The first fastener 78 may comprise a plurality of apertures 86 and the second fastener 82 may comprise a buckle 88, as shown in FIGS. 4 and 5. The buckle 88 is frame type so that a prong 90 of the buckle 88 is positioned to be selectively inserted into a respective aperture 86 to couple the band 66 to the wrist of the user. The first fastener 78 and the second fastener 82 may comprise other fastening means, such as, but not limited to, hook and loop fasteners, clasps, and the like.

In use, the strap 36 is positioned around a neck of the user and the set of first sensors 26 is adhesively coupled to the chest of the user. The sleeve 48 is positioned over the digit of the hand of the user so that the second sensor 46 contacts the skin of the digit. The band 66 is coupled to the wrist of the user so that the third sensor 58 contacts the skin of the wrist. The user is then positioned to view measurements of cardiovascular parameters in real time and to view graphical and animated representations of the cardiovascular parameters over time on the display 20 of the electronic device 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A cardiovascular monitoring assembly comprising:
a set of transceivers configured for wireless communication;
a set of detectors configured for coupling to a user, the detectors being optical type, each detector being configured for measuring a respective cardiovascular parameter such that the set of detectors is configured for measuring a variety of cardiovascular parameters, each detector being operationally coupled to a respective transceiver such that the detector is positioned for relaying a measurement of an associated cardiovascular parameter to the respective transceiver wherein the respective transceiver is configured for transmitting the measurement to an electronic device; and
processing programming code positioned on the electronic device enabling the electronic device for displaying the measurements of the cardiovascular parameters on a display of the electronic device and for logging the measurements in a data storage module of the electronic device.

2. The assembly of claim 1, further including the set of detectors comprising:
a set of first sensors configured for adhesively coupling to skin of the user wherein each first sensor is configured for measuring a pulse rate of the user;
a second sensor configured for coupling to an extremity of the user wherein the second sensor is configured for measuring a blood oxygenation level of the user; and
a third sensor configured for coupling to at least one of a limb and a digit of a hand of the user wherein the third sensor is configured for measuring a blood pressure of the user.

3. The assembly of claim 1, further including the set of first sensors comprising three first sensors.

4. The assembly of claim 2, further comprising:
a communications housing defining an interior space, an associated transceiver being coupled to the communications housing and positioned in the interior space, the set of first sensors being wiredly coupled to the communications housing;
a communications battery coupled to the communications housing and positioned in the interior space; and
a communications microprocessor coupled to the communications housing and positioned in the interior space, the communications microprocessor being operationally coupled to the communications battery, the set of first sensors, and the associated transceiver such that the communications microprocessor is positioned for receiving pulse rate measurements from the set of first sensors and for actuating the associated transceiver for transmitting the measurements to the electronic device.

5. The assembly of claim 4, further including a strap having opposing ends coupled to the communications housing defining a loop wherein the strap is configured for positioning around a neck of the user such that the communications housing is positioned proximate to a sternum of the user and such that the set of first sensors is positioned for adhesively coupling to the chest of the user.

6. The assembly of claim 4, further including a switch coupled to the communications housing, the switch being operationally coupled to the communications microprocessor wherein the switch is configured for switching for signaling the communications microprocessor for denoting a pulse rate measurement of interest.

7. The assembly of claim 6, further including the switch comprising a button, the button being depressible.

8. The assembly of claim 7, further including the button being red colored.

9. The assembly of claim 2, further comprising:
a sleeve configured for inserting the digit of the hand of the user, the second sensor being coupled to the sleeve wherein the second sensor is configured for contacting skin of the digit of the hand as the digit is inserted into the sleeve;
a transmissions housing coupled to the sleeve, the transmissions housing defining an internal space, an associated transceiver being coupled to the transmissions housing and positioned in the internal space;
a transmissions battery coupled to the transmissions housing and positioned in the internal space; and
a transmissions microprocessor coupled to the transmissions housing and positioned in the internal space, the transmissions microprocessor being operationally coupled to the transmissions battery, the second sensor, and the associated transceiver such that the transmissions microprocessor is positioned for receiving the measurement of the blood oxygenation level from the second sensor and for actuating the associated transceiver for transmitting the measurement to the electronic device.

10. The assembly of claim 2, further comprising:
a wrist cuff comprising a cuff housing and a band wherein the band is configured for coupling the cuff housing to a wrist of the user, the cuff housing defining an inner space, an associated transceiver being coupled to the cuff housing and positioned in the inner space, the third sensor being coupled to a lower face of the cuff housing wherein the third sensor is configured for contacting skin of the wrist of the user;
a cuff battery coupled to the cuff housing and positioned in the inner space; and
a cuff microprocessor coupled to the cuff housing and positioned in the inner space, the cuff microprocessor being operationally coupled to the cuff battery, the third sensor, and the associated transceiver such that the cuff microprocessor is positioned for receiving the measurement of the blood pressure from the third sensor and for actuating the associated transceiver for transmitting the measurement to the electronic device.

11. The assembly of claim 10, further comprising:
a first fastener coupled to the band proximate to a first end of the band; and
a second fastener coupled to the band proximate to a second end of the band, the second fastener being complementary to the first fastener such that the second fastener is positioned for selectively coupling to the first fastener for coupling the band around the wrist of the user.

12. The assembly of claim 11, further including the first fastener comprising a plurality of apertures and the second fastener comprising a buckle, the buckle being frame type such that a prong of the buckle is positioned for selectively inserting into a respective aperture for coupling the band to the wrist of the user.

13. The assembly of claim 10, further including a screen coupled to an upper face of the cuff housing, the screen being operationally coupled to the cuff microprocessor such that the cuff microprocessor is positioned for selectively actuating the screen for displaying a time and the measurement of the blood pressure. parameters.

14. The assembly of claim 1, further including command programming code positioned on the electronic device enabling the electronic device for selectively signaling the set of transceivers for actuating the set of detectors for obtaining the measurements of the cardiovascular

15. A cardiovascular monitoring assembly and electronic device combination comprising:
an electronic device;
a set of transceivers configured for wireless communication with the electronic device;
a set of detectors configured for coupling to a user, the detectors being optical type, each detector being configured for measuring a respective cardiovascular parameter such that the set of detectors is configured for measuring a variety of cardiovascular parameters, each detector being operationally coupled to a respective transceiver such that the detector is positioned for relaying a measurement of an associated cardiovascular parameter to the respective transceiver wherein the respective transceiver is configured for transmitting the measurement to the electronic device; and
processing programming code positioned on the electronic device enabling the electronic device for displaying the measurements of the cardiovascular parameters on a display of the electronic device and for logging the measurements in a data storage module of the electronic device.

16. A cardiovascular monitoring assembly comprising:
a set of transceivers configured for wireless communication;
a set of detectors configured for coupling to a user, the detectors being optical type, each detector being configured for measuring a respective cardiovascular parameter such that the set of detectors is configured for measuring a variety of cardiovascular parameters, each detector being operationally coupled to a respective transceiver such that the detector is positioned for relaying a measurement of an associated cardiovascular parameter to the respective transceiver wherein the respective transceiver is configured for transmitting the measurement to an electronic device, the set of detectors comprising:
a set of first sensors configured for adhesively coupling to skin of the user wherein each first sensor is configured for measuring a pulse rate of the user, the set of first sensors comprising three first sensors,
a second sensor configured for coupling to an extremity of the user wherein the second sensor is configured for measuring a blood oxygenation level of the user, and
a third sensor configured for coupling to at least one of a limb and a digit of a hand of the user wherein the third sensor is configured for measuring a blood pressure of the user;
a communications housing defining an interior space, an associated transceiver being coupled to the communications housing and positioned in the interior space, the set of first sensors being wiredly coupled to the communications housing;
a strap having opposing ends coupled to the communications housing defining a loop wherein the strap is configured for positioning around a neck of the user such that the communications housing is positioned proximate to a sternum of the user and such that the set of first sensors is positioned for adhesively coupling to the chest of the user;
a communications battery coupled to the communications housing and positioned in the interior space;
a communications microprocessor coupled to the communications housing and positioned in the interior space, the communications microprocessor being operationally coupled to the communications battery, the set of first sensors, and the associated transceiver such that the communications microprocessor is positioned for receiving pulse rate measurements from the set of first sensors and for actuating the associated transceiver for transmitting the measurements to the electronic device;
a switch coupled to the communications housing, the switch being operationally coupled to the communications microprocessor wherein the switch is configured for switching for signaling the communications microprocessor for denoting a pulse rate measurement of interest, the switch comprising a button, the button being depressible, the button being red colored;
a sleeve configured for inserting the digit of the hand of the user, the second sensor being coupled to the sleeve wherein the second sensor is configured for contacting skin of the digit of the hand as the digit is inserted into the sleeve;
a transmissions housing coupled to the sleeve, the transmissions housing defining an internal space, an associated transceiver being coupled to the transmissions housing and positioned in the internal space;
a transmissions battery coupled to the transmissions housing and positioned in the internal space;
a transmissions microprocessor coupled to the transmissions housing and positioned in the internal space, the transmissions microprocessor being operationally coupled to the transmissions battery, the second sensor, and the associated transceiver such that the transmissions microprocessor is positioned for receiving the measurement of the blood oxygenation level from the second sensor and for actuating the associated transceiver for transmitting the measurement to the electronic device;
a wrist cuff comprising a cuff housing and a band wherein the band is configured for coupling the cuff housing to a wrist of the user, the cuff housing defining an inner space, an associated transceiver being coupled to the cuff housing and positioned in the inner space, the third sensor being coupled to a lower face of the cuff housing wherein the third sensor is configured for contacting skin of the wrist of the user;
a first fastener coupled to the band proximate to a first end of the band;
a second fastener coupled to the band proximate to a second end of the band, the second fastener being complementary to the first fastener such that the second fastener is positioned for selectively coupling to the first fastener for coupling the band around the wrist of the user, the first fastener comprising a plurality of apertures and the second fastener comprising a buckle, the buckle being frame type such that a prong of the buckle is positioned for selectively inserting into a respective aperture for coupling the band to the wrist of the user;
a cuff battery coupled to the cuff housing and positioned in the inner space;
a cuff microprocessor coupled to the cuff housing and positioned in the inner space, the cuff microprocessor being operationally coupled to the cuff battery, the third sensor, and the associated transceiver such that the cuff microprocessor is positioned for receiving the measurement of the blood pressure from the third sensor and for actuating the associated transceiver for transmitting the measurement to the electronic device;
a screen coupled to an upper face of the cuff housing, the screen being operationally coupled to the cuff microprocessor such that the cuff microprocessor is positioned for selectively actuating the screen for displaying a time and the measurement of the blood pressure;
processing programming code positioned on the electronic device enabling the electronic device for displaying the measurements of the cardiovascular parameters on a display of the electronic device and for logging the measurements in a data storage module of the electronic device; and
command programming code positioned on the electronic device enabling the electronic device for selectively signaling the set of transceivers for actuating the set of detectors for obtaining the measurements of the cardiovascular parameters.
